(19) United States
(12) Patent Application Publication
(10) Pub. No.: US 2009/0258878 A1
(43) Pub. Date: Oct. 15, 2009

(54) TRIAZINE DERIVATIVE COMPOUND AND COMPOSITION FOR TREATING CANCER CONTAINING THEM

(76) Inventors: Hyun-Kee Kim, Seoul (KR); Jin-Woo Kim, Seoul (KR)

Correspondence Address: ROBERT E. BUSHNELL & LAW FIRM, 2029 K STREET NW, SUITE 600, WASHINGTON, DC, 20006-1004, US

(21) Appl. No.: 12/309,111

(22) PCT Filed: Jul. 6, 2007
(86) PCT No.: PCT/KR2007/003296

§ 371 Date: Feb. 2, 2009

(30) Foreign Application Priority Data
Jul. 7, 2006 (KR) 10-2006-0064059

(51) Int. Cl. A61K 31/53; C07D 251/18; A61P 35/00

(52) U.S. Cl. 514/245; 544/180

(57) ABSTRACT

The present invention relates to a Triazine derivative compound, pharmaceutically acceptable salt thereof and a pharmaceutical composition containing them. Compound or composition of the present invention is useful for cancer treatment, and especially has superior effect in breast cancer treatment.

### TECHNICAL FIELD

The present invention relates to new compound useful for treating cancer, particularly, for treating breast cancer, and composition containing them, and a treatment method using them.

### BACKGROUND ART

According to recent local reports, they are analyzing that attack rate of chronic disease such as hypertension, cerebrovascular disease, diabetes, etc. as well as cancer has continuously increased, thereby economic loss reaches to approximately KRW 37 trillion. Especially, cancer is the first leading cause of death rate by diseases in Korea, and cancer pain affects not only considerable economic costs but also mental loss. However, according to the progress of cancer treatment, the number of cancer deaths is continuously decreasing, however, the number of cancer patients per se is continuously increasing and administration period of treatment drugs becomes lengthy.

Meanwhile, as breast cancer arises from breast, and any one of many kinds of cells in breast can be developed to cancer so that there are so many kinds of breast cancer. However, since most of breast cancer arises from cells in both of duct and lobe, among them, from duct cell, breast cancer generally refers to cancer arose from epithelial cell of duct and lobe. Although breast cancer is one of carcinomas on which the most research have been made, there is not still clearly demonstrated as an initiator of breast cancer.

As breast cancer was the fifth of all cancers following stomach cancer, lung cancer, live cancer and colorectum cancer in Korea, its incidence rate was 6.5% (5,444 patients) of all cancer in 2000, and ranked the second most common cancer following stomach cancer of female malignant cancers. However, in 2002 its incidence rate was 7.4% (7,359 patients) and breast cancer occupied the first ranking (16.8%) of female malignant cancers replacing stomach cancer (National Cancer Center, research report of 2005). Meanwhile, breast cancer occurs to women mostly; however, it can occur to men as well with the incidence rate reckoned at 1/100 lower than that of women.

Among current anti-cancer therapeutics, anti-cancer agents using chemotherapy are occupying approximately 30% thereof, and as an example, more than 40 to 50 sorts of anti-agents including Lupron for prostate cancer; Taxol for ovarian cancer; and Zoladex for breast cancer are used in the clinic, other chemotherapy agents are under development. The development object of anti-cancer agents is to overcome toxic side effects, induce of tolerance and relapse at the same time conquering cancers selectively and fundamentally. Especially, since breast cancer death rate in conventional treatment methods was very high, the need of treating breast cancer is desperately requested.

### SUMMARY OF THE INVENTION

Accordingly, the object of the present invention is to provide new compounds useful for treating cancer, a composition containing them, and a treatment method using them.

To achieve the above object, the present invention provides a triazine derivative compound of formula (I),

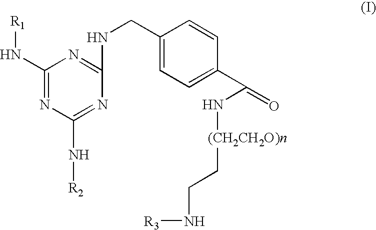

pharmaceutically acceptable salt thereof, a composition for treating cancer them, and a method for treating cancer, wherein in Formula (I) R₁ is linear or branched alkyl of carbon number 1 to 8, R₂ is any one selected from a group consisting of linear branched alkyl, allyl, pyridinylmethyl, pyridinylethyl and pyridinylpropyl of carbon number 1 to 8, R₃ is a non-substituted benzoyl or substituted benzoyl by alkyl of carbon number 1 to 3, and n is integer of 1 to 3.

Preferably, in Formula (I), R₁ is butyl, R₂ is any one selected from a group consisting of allyl, 1-ethylpropyl and pyridinylmethyl, R₃ is a non-substituted benzoyl, and n is 2, and more preferably, in Formula (I), R₂ is allyl.

### BRIEF DESCRIPTION OF THE DRAWINGS

The accompanying drawings, which are included to provide a further understanding of the invention and are incorporated in and constitute a part of this application, illustrate embodiments of the invention and together with the description serve to explain the principle of the invention. In the drawings:

FIG. 1 are photos magnified by 200 times showing that compounds according to an Example of the present invention inhibit growth of MCF-7 breast cancer cell, respectively;

FIG. 2 is a graph showing degree of growth inhibition after treating the MCF-7 breast cancer cells with the compound of 100 μM according to an Example of the present invention;

FIG. 3 is, as an experimental result for a negative control group, a photo showing breast cancer mass induced by injecting MCF-7 breast cancer cells into nude mouse as a result of negative control group;

FIG. 4 are photos showing the changes of mass size after administrating the Dan-BC1 compound according to an Example of the present invention into breast cancer mass induced by injecting breast cancer cells into nude mouse, respectively;

FIG. 5 are photos showing the changes of mass size after administrating the Dan-BC2 compound according to an Example of the present invention into breast cancer mass induced by injecting breast cancer cells into nude mouse, respectively;

FIG. 6 are photos showing the changes of mass size after administrating the Dan-BC3 compound according to an Example of the present invention into breast cancer mass induced by injecting breast cancer cells into nude mouse, respectively;

FIG. 7 is a graph comparing tumor inhibition effects of Dan-BC1, Dan-BC2 and Dan-BC3 triazine derivative compound according to an Example of the present invention in nude mouse into which breast cancer is induced;

FIG. 8 are photos showing the changes of mass size after administrating the Dan-BC1 compound according to an Example of the present invention into breast cancer mass induced naturally in a transgenic animal model of cancer formation;

FIG. 9 are photos showing the changes of mass size after administrating the Dan-BC2 compound according to an Example of the present invention into breast cancer mass induced naturally in a transgenic animal model of cancer formation;

FIG. 10 are photos showing the changes of mass size after administrating the Dan-BC3 compound according to an Example of the present invention into breast cancer mass induced naturally in a transgenic animal model of cancer formation;

FIG. 11 is a graph comparing tumor inhibition effects of Dan-BC1, Dan-BC2 and Dan-BC3 Triazine derivative compound according to an Example of the present invention in a transgenic animal model of cancer formation.

### DETAILED DESCRIPTION OF THE INVENTION

Thereafter, more details regarding a new cancer treatment agent according to the present invention, a composition containing them and a method for treating cancer using them will be described.

The present invention is based on the fact that triazine derivative compounds indicated by formula (I) shows excellent effect in treating cancers, especially, breast cancer. Triazine derivative compound library is originally designed from myoseverin derivatives by the known structure activity relationship SAR data and computer aided modeling to develop myoseverin-pseudo anti-tublin drugs (see: Moon, H-S., et al., J Am Chem. Soc., 124(39), 11608-11609 (2002)). Through an inspection on the effectiveness of the anti-cancer compounds found using a series of cancer cells containing breast cancer cells cultivated using such triazine derivative compounds library, it was confirmed that triazine derivative compounds and its pharmaceutically acceptable salt showed excellent effects in treating cancer, especially, breast cancer.

Additionally, among the triazine derivative compounds indicated formula (I), the compound Dan-BC1 (C₃₄H₄₂N₈O₄, molecular weight: 590.724) indicated by the following formula (II), the compound Dan-BC2(C₃₄H₄₃N₉O₄, molecular weight: 641.772) indicated by the following formula (III), and the compound Dan-BC3(C₃₃H₄₈N₈O₄, molecular weight: 620.793) indicated by the following formula (IV) are more effective in the treatment of cancers in various aspects such as cancer suppression effect, etc.

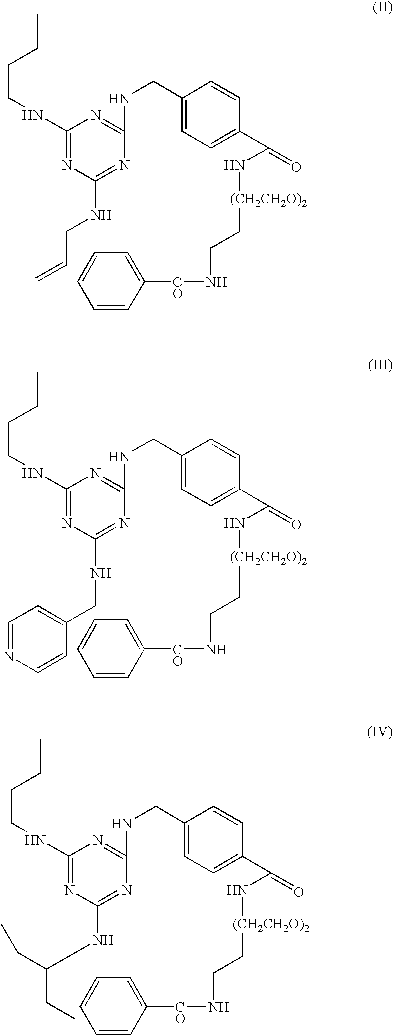

To find out anti-cancer effects of the found triazine derivative compounds, chemical and genetic screenings were performed in breast cancer cells, colorectum cancer cells, kidney cancer cells, brain cancer cells, etc., and it was demonstrated that the compounds of formula (I) in the above triazine derivative library, more preferably, the three compounds, Dan-BC1, Dan-BC2 and Dan-BC3 were more useful in terms of their cancer cell suppression activity.

More precisely, the compounds according to the present invention showed the strongest growth inhibition effects on cells in MCF-7 breast cancer cell line (ATCC Number: HTB-22), and next, with an order of MDA-MB-435 (ATCC Number: HTB-129) breast cancer cell line, MDA-N breast cancer cell line (Wang, J. J., et al., Anticancer Drugs, 15(3), 277-286 (2004)). These compounds also showed the growth inhibition effect of cancer cells in HCT15 (ATCC Number: CCL-225) colorectum cancer cell line, NCl/ADR-RES breast cancer cell line(Ehrlichova, M., et al., Anticancer Res., 25(6B), 4215-4224 (2005)), CAKI-1 (ATCC Number: HTB-46) kidney cancer cell line, ACHN (ATCC Number: CRL-1611) kidney cancer cell line, SF295 brain cancer cell line (Rao, V. K., et al., Cancer Genet Cytogenet, 160(2), 126-133 (2005)) and UO-31 kidney cancer cell line (Jansen, A. P., et al., Mol, Cancer Ther., 3(2), 103-110 (2004); Chen, Y-L., et al., Eur J Med. Chem., 40, 928-934 (2005)) as well.

IC₅₀ value of the triazine derivative compounds according to the present invention is about 10 μM, their active concentration is about 1 to 10 μM, and Cytotoxicity was not observed up to about 100 μM, which can be varied depending on the used cell line and kinds of concrete compounds.

“Pharmaceutically acceptable salt” of the present invention refers to a salt produced by non-toxic or little toxic acids or bases. In case where the compounds of the present invention are comparatively acidic, base addition salts can be obtained by contacting neutral form of the compounds with enough desired base and proper inert solvent. Pharmaceutically acceptable base addition salt includes, but is not limited to, salts such as natrium, kalium, calcium, ammonium, magnesium or organic amino. In case where the compounds of the present invention are comparatively basic, acid addition salts can be obtained by contacting neutral form of the compounds with enough desired acid and proper inert solvent. Pharmaceutically acceptable acid addition salt includes, but is not limited to, salts such as propionic acid, isobutylic acid, oxalic acid, malic acid, malonic acid, benzoic acid, succinic acid, suberic acid, fumaric acid, mandelic acid, phthalic acid, benzenesulfonic acid, p-tolylsulfonic acid (PTSA), citric acid, tartaric acid, methansulfonic acid, hydrochloric acid, bromic acid, nitric acid, carbonic acid, monohydrogencarbonic acid, phosphoric acid, monohydrogen phosphate, dihydrogen phosphate, sulfuric acid, monohydrogen sulfate, hydrogen iodide, phosphorous acid, etc., but they not limited thereto. Also, it includes salts of amino acid such as arginate and analogues of organic acid such as glucuronic or galactunoric acids.

Some compounds of the present invention includes hydrated form and thus may exist in both a solvated and an unsolvated forms. Some compounds of the present invention may exist in both crystal and amorphorous forms. Such all physical forms are included within the scope of the present invention. Also, some compounds of the present invention can have asymmetric carbon atoms of optical center or double bonds and thus, racemate, enantiomer, diastereomer and geometrical isomer, etc. may exist, they also are included within the scope of the present invention.

The present invention also provides a pharmaceutical composition comprising a triazine derivative compound according to the present invention or pharmaceutically acceptable salt thereof, and pharmaceutically accepted vehicle or additive. Triazine derivative compound of the present invention or pharmaceutically acceptable salt thereof may be administered independently or in combination with a convenient carrier, vehicle, etc. Dosage form of such administration can be a single dosage form or repeated dosage form.

The composition of the present invention may be, but not limed to, solid or liquid type preparations, and solid type preparations are acids, granules, tablets, capsules, suppository, etc. A solid type preparation can contain vehicle, flavouring agents, binder, antiseptics, disintergrant, lubricant, filler, etc., but they are not limited thereto. As liquid type preparations, there are solution agent such as water, propylene glycol solution, suspending liquid agent, emulsifier, etc., but they are not limited thereto, and can be produced by adding coloring agent, flavouring agent, stabilizing agent, viscosifying agent, etc.

The composition of the present invention can be administrated, depending on disease to be treated and the subject condition, as entecavir, administration agent (for example, intramuscular administration, intraperitoneal administration, intravenous administration, infusion, subcutaneous administration, implant), inhaler, intranasal administration drugs, vaginal agent, rectal administration drugs, sublingual preparation, transdermal drug, topical drug, etc., but they are not limited thereto. According to administration route, it can be formulated as a proper dosage unit preparation form including carrier, additive and vehicle, which are usually used, non-toxic and pharmaceutically acceptable. Depot preparation form which can sustainedly release drugs for a certain time is also included within the scope of the present invention.

Triazine derivative compound of the present invention or pharmaceutically acceptable salt thereof may be administrated about 0.04 mg/kg to about 20 mg/kg per day, dose of about 0.4 mg/kg to about 2 mg/kg per a day is preferable. However, the dose can be varied depending on patient's condition (age, sex, weight, etc.), severity of treating condition, used compound, etc. If necessary, entire dose per day are divided for convenience, and can be administrated several times over a day.

Hereinafter, in order to describe the present invention more specifically, it is referred to the following Examples. However, the Examples according to the present invention can be modified into various forms, and the scope of the present invention should not be interpreted as being limited to the Examples as described below. The Examples of the present invention are provided exemplarily to help specific understanding.

### Example 1

### Design and Synthesis of Triazine Derivative Compound Library

Design and synthesis of triazine derivative compound library based on triazine structure were performed on the basis of a method (J Am Chem. Soc., 125, 11804-11805 (2003)) disclosed by Khersonsky, S. M., etc. Briefly, in order to produce triazine derivative compound library, solid-phase method was used, three kinds of building blocks were produced separately, and they generated 1,536 pure triazine derivative compounds by being connected orthogonally.

More specifically, as shown in the following reaction 1, three kinds of building blocks are (I) PAL-resin binding primary amine, (II) 4,6-dichloro-[1,3,5] triazine to which amine, alcohol or thiol is attached in position 2, and (III) a series of primary or secondary amine. Building block I was generated by the primary amine containing various benzylamine being attached to PAL aldehyde resine using reductive amination. Also, building block II was produced by introducing alkyl/aryl alcohol, bulky amine or aniline having a low nucleophilic reactivity by using a high reactivity of the first substitution reaction on triazine trichloride. Various primary and the secondary amines were prepared in advance for building block III.

Through the simple synthesis processes using the building blocks which was previously produced or available commercially, triazine derivative compounds of the present invention were produced, and all produced compounds were confirmed as LC-MS, and the purity thereof was more than 98%.

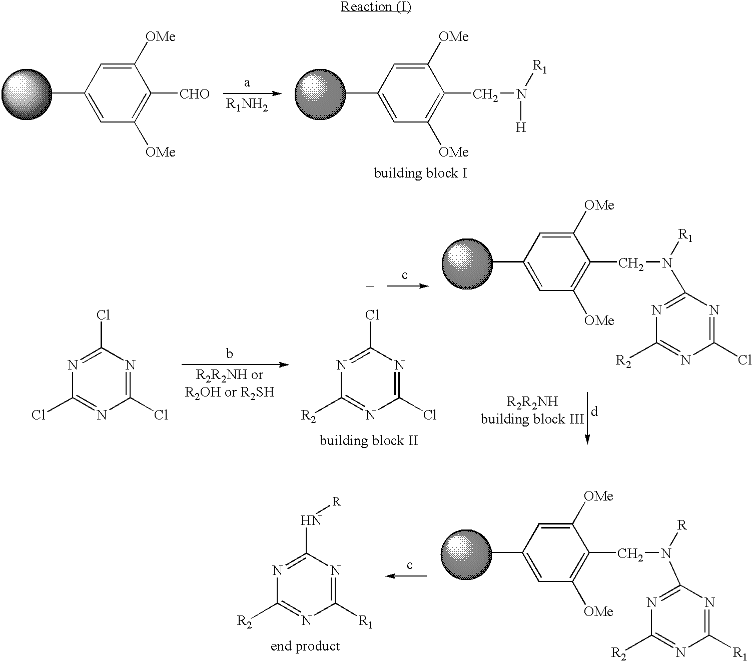

(a) R₁NH₂ (5 equivalent weight), 2% acetic acid in THF, room temperature, 1 hour, thereafter, NaB(OAc)₃H (7 equivalent weight), room temperature, 12 hours.
(b) R₂R₂′NH, R₂OH or R₂SH in THF (1 equivalent weight), 0° C. 1 hour.
(c) Building block II in THF (4 equivalent weight), DIEA (4 equivalent weight), 60° C. 3 hours.
(d) R₃R₃′NH (4 equivalent weight) in the 1:1 solvent mixture of NMP:n-BuOH, DIEA (4 equivalent weight), 120° C. 3 hours.
(e) 5% TFA in DCM, 10 minutes.

### Example 2

### Cell Culture

Cancer cells of MCF-7 breast cancer cell line (ATCC Number: HTB-22), MDA-MB-435 (ATCC Number: HTB-129) breast cancer cell line, MDA-N breast cancer cell line (Wang, J. J., et al., Anticancer Drugs, 15(3), 277-286 (2004), HCT15 (ATCC Number: CCL-225) colorectum cancer cell line, NCI/ADR-RES breast cancer cell line (Ehrlichova, M., et al., Anticancer Res., 25(6B), 4215-4224 (2005), CAKI-1 (ATCC Number: HTB-46) kidney cancer cell line, ACHN (ATCC Number: CRL-1611) kidney cancer cell line, SF295 brain cancer cell line (Rao, V. K., et al., Cancer Genet Cytogenet, 160(2), 126-133 (2005)) and UO-31 kidney cancer cell line (Jansen, A. P., et al., Mol. Cancer. Ther., 3(2), 103-110 (2004); Chen, Y-L., et al., Eur J Med. Chem., 40, 928-934 (2005)) were cultivated in an incubation at 37° C. 5% CO₂. During culture and subculture, the cancer cells were grown in DMEM containing 10% fetal bovine serum.

### Example 3-1

### Screening of Triazine Derivative Compounds Using Cancer Cells

The lyophilized library compounds were resuspended upto the final concentration of 10 mM by resuspending with dimethyl sulfoxide (DMSO). The cancer cells were placed in 24-well plate to be 5×10⁴ per well with 1 ml culture media one day before adding library compound. Every compound was to added to be final concentration which was indicated. In each experiment, DMSO was used as negative control. Cells were cultivated for 72 hours, unless other indicated, and the survival rate of cells was measured using CellTilter 96 aqueous non-radioactive cell proliferation assay (Promega, Madison, Wis., USA).

CellTiter 96™ Aqueous non-radioactive cell proliferation assay was an uniform and non-colorimetric method to measure the number of surviving cells in assays of cell toxification, cell proliferation or chemosensitivity. CellTiter 96™ Aqueous assay was composed of tetrazolium compound (3-(4,5-dimethyazol-2-yl)-5-(3-caboximetoxipenyl)-2-(4-sulpopenyl)-2H-tetrazolium, inner salt, MTS) and the electron coupling indicator (phenazine methosulfate, PMS). MTS was bio-reduced by cells which were solved into a system culture media into which a formazan product solved. The 490 nm absorbance of formazan product could directly be measured without additional process from 96-well assay plate. Transformation of MTS into aqueous formazan product was performed by dehydrogenase enzyme found in the metabolically active cells. The quantity of water-soluble Formazan product was directly proportion to the number of surviving cells in culture solution which was measured by the quantity of 490 nm absorbance intensity.

### Example 3-2

### Assessing the Effect of Compound Dan-BC1, Dan-BC2 or Dan-BC3 on Viability of Breast Cell Line

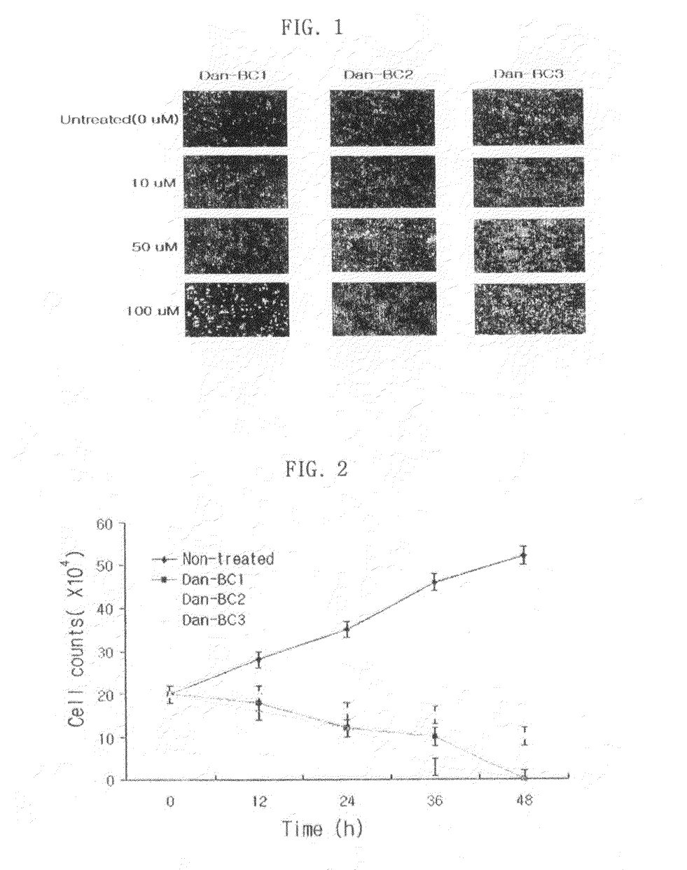

After administration of each compound of various concentration into MCF-7 breast cancer cell line, the active of breast cancer cell was observed. 2×10⁵ MCF-7 cells were respectively distributed into each 6 well plates and grown. Thereafter, Dan-BC1, Dan-BC2 and Dan-BC3 compounds were diluted to be dose concentration of 10, 50 and 100 μM, and after treating them into cancers for 2 days according to the concentration, they were compared by measuring the cell viability with those of drug-untreated group. The results were shown in FIG. 1 and FIG. 2.

In FIG. 1, a photo (magnification of 200 times) was shown in which said 3 kinds of compounds inhibit growth of MCF-7 breast cancer cell. As shown in FIG. 1, all of Dan-BC1, Dan-BC2 and Dan-BC3 showed weak growth inhibition effect on cell at 10 μM concentration and no significant growth inhibition effect on cell even at 50 μM concentration, but at 100 μM concentration, an extinct effect on cell was apparent to the naked eye. Accordingly, we proceeded with the follow-up experiments with the 100 μM administration concentration of each compound of Triazine derivatives.

In FIG. 2, degree of growth inhibition after MCF-7 breast cancer cell was treated by 100 μM of said 3 kinds of compound was shown.

Growth inhibition effect of Dan-BC1, Dan-BC2 and Dan-BC3 against breast cancer cell was tested and the results showed that about 60% of the treated cells survived at 12 hours after administration of 100 μM of Dan-BC3 compared with cells of breast cancer cell line which had no administration of pharmaceutical preparations, only about 37% of the treated cells survived at 24 hours compared with the cells of breast cancer cell line which had no administration of pharmaceutical preparations, and about 7% of the cells survived at 36 hours, and at 48 hours, all cells reached extinct.

As for Dan-BC1, about 68% of cells survived at 12 hours after administration of 100 μM of the compound, only about 37% of cells survived at 24 hours compared with cells of breast cancer cell line which had no administration of pharmaceutical preparations, about 27% of cells survived at 36 hours, and at 48 hours, all cells reached extinction.

As for Dan-BC2, about 75% of cells survived at 12 hours after administration of 100 μM of the compound compared with the cells of breast cancer cell line which had no administration of pharmaceutical preparations, only about 49% of the cells survived at 24 hours compared with the cells of breast cancer cell line which had no administration of pharmaceutical preparations, about 33% survived at 36 hours and only 20% survived at 48 hours.

### Example 4-1

### Formation of Breast Cancer Mass in Nude Mouse and Assessment of Experiment Compound

In order to measure the capacity for tumor formation of MCF-7 tumor cell in nude mouse, 30 female nude mice aged 4-5 weeks after birth (BALB/c line athymic nu/nu) were subcutaneously injected with 5×10⁶ MCF-7 tumor cells in 200 μl of PBS, which has the cell viability more than 95% by trypan blue dye exclusion inspection, and thereafter, when the diameter of tumor formed after 2 weeks to 2 months was longer than 10-25 mm, the administration of experiment compound has begun.

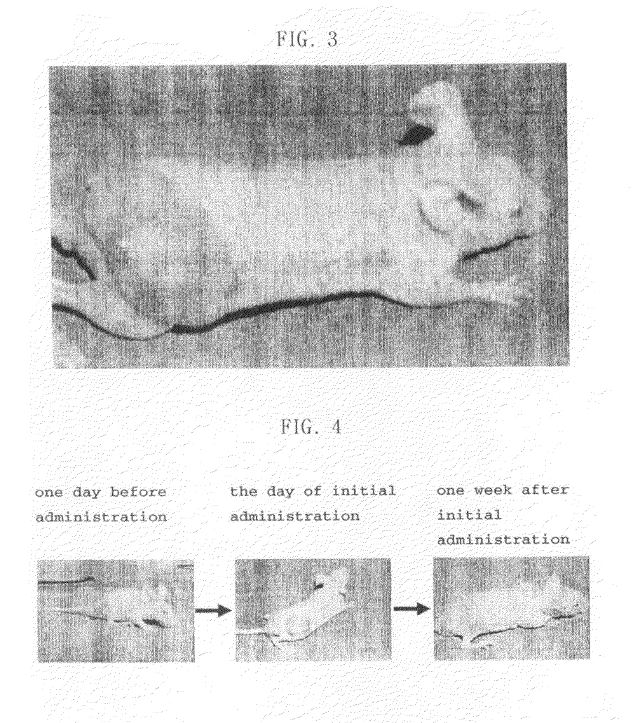

Weekly administration of experiment compound was initiated 2 days after measurement of tumor size. The concentration of the first administration was 500 μM, respectively. Photos of nude mice formed cancer to which experiment compound has not been administered were shown in FIG. 3, in which the tumor size was 25 mm in major axis, 20 mm in minor axis, 15 mm height, and 27 g weight.

### Example 4-2

### Assessment of In Vivo Breast Cancer Inhibition Effect of Each Compound

Similarly to Example 4-1, after formation of breast cancer in nude mouse, the change of mass size was observed after administrating Triazine derivative compounds of the present invention, Dan-BC1, Dan-BC2 and Dan-BC3.

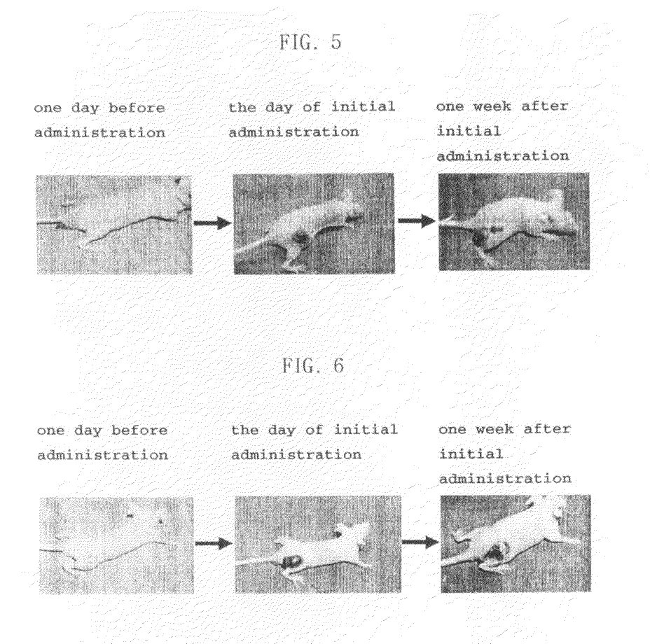

After administration of Dan-BC1, Dan-BC2 and Dan-BC3 compounds two times by 500 μM, as a result of comparison with the size of breast cancer mass (25 mm major axis, 20 mm minor axis, 15 mm height) of negative control group of the foregoing Example 4-1, the strong in vivo anticancer effect of Triazine derivatives of the present invention regarding breast cancer formed in nude mouse was confirmed by mass size comparison after administration of Dan-BC1, Dan-BC2 and Dan-BC3, which was decreased to an extent described in the following tables 1, 2 and 3, respectively. Each photo is shown in FIGS. 4, 5 and 6.

TABLE 1

| | Drug | Major | Minor | | |
|---|---|---|---|---|---|
| Compound | Concentration | Axis | Axis | Height | Weight |
| Dan-BC1 | (μM) | (mm) | (mm) | (mm) | (g) |
| 1 day before | — | 4 | 2 | 2 | 18.5 |
| First | | | | | |
| Administration | | | | | |
| the day of | 500 | 12 | 10 | 5 | 21.2 |
| First | | | | | |
| Administration | | | | | |
| 1 week after | 500 | 10 | 8 | 2 | 19.5 |
| First | | | | | |
| Administration | | | | | |

TABLE 2

| | Drug | Major | Minor | | |
|---|---|---|---|---|---|
| Compound | Concentration | Axis | Axis | Height | Weight |
| Dan-BC2 | (μM) | (mm) | (mm) | (mm) | (g) |
| 1 day before | — | 4 | 2 | 2 | 19.5 |
| First | | | | | |
| Administration | | | | | |
| the day of | 500 | 12 | 10 | 5 | 21.2 |
| First | | | | | |
| Administration | | | | | |
| 1 week after | 500 | 15 | 10 | 3 | 20.5 |
| First | | | | | |
| Administration | | | | | |

TABLE 3

| | Drug | Major | Minor | | |
|---|---|---|---|---|---|
| Compound | Concentration | Axis | Axis | Height | Weight |
| Dan-BC3 | (μM) | (mm) | (mm) | (mm) | (g) |
| 1 day before | — | 4 | 2 | 2 | 19.5 |
| First | | | | | |
| Administration | | | | | |
| the day of | 500 | 12 | 7 | 5 | 21.2 |
| First | | | | | |
| Administration | | | | | |
| 1 week after | 500 | 15 | 10 | 2 | 20.5 |
| First | | | | | |
| Administration | | | | | |

The results of the foregoing tables 1, 2 and 3 are represented in FIG. 7. As shown in FIG. 7, an extinction effect of each compound on breast cancer cell in nude mouse was high in case of Dan-BC1, Dan-BC3 compound, and Dan-BC2 compound in that order.

### Example 5-1

### Assessment of Anti-Cancer Effect of Dan-BC1, Dan-BC2 and Dan-BC3 Compounds Using Animal Model Transformed to Form Cancer

Anticancer effect of Dan-BC1, Dan-BC2 and Dan-BC3 according to the present invention was assessed using the mouse transformed with human proto-oncogene causing cancer which was over-expressed in mammal.

Since the cell line used for the transformed mouse has capacity for forming tumor, it forms a carcinoma such as palpable tumor when transplanted into mammal. Cell line used in the present experiment produces a large quantity of HCCR-1 protein by overexpressing human proto-oncogene (hereafter, HCCR-1), which protein interacts with D52 protein (GenBank Reg. No. NM003288) as reported as being related to cancer (Byrne, J. A. et al., Cancer Res., 55, 2896-2903 (1995); and Bryne, J. A. et al., Oncogene, 16, 873-881 (1998) reference), activates protein Kinases C and Telomerase, incapacitates the function of controlling cell cycle checkpoint, and transforms into malignant tumor though the course of lowering the expression of egr-1 gene.

Especially, in this course, although the expression of tumor inhibition gene p53 related with cell cycle control increases, the cells seem to be transformed into malignant tumor since most of produced p53 protein was inactivated and does not function for tumor inhibition.

Namely, HCCR-1 is regarded as inactivating the function of tumor inhibition gene p53 (refer to Korean Patent Reg. No. 426-452 “character transformed mammal into human proto-oncogene and diagnostic kit for breast cancer, kidney cancer, ovarian cancer or stomach cancer”; Korean Patent Reg. No. 367978 “human cervical cancer 1 proto-oncogene and protein coded hereby”).

Therefore, since such transformed mammal forms cancer by overexpressing of human proto-oncogene HCCR-1, it can be useful for detection of anticancer materials, etc., especially for screening of drugs effective against breast cancer, kidney cancer, ovarian cancer or stomach cancer.

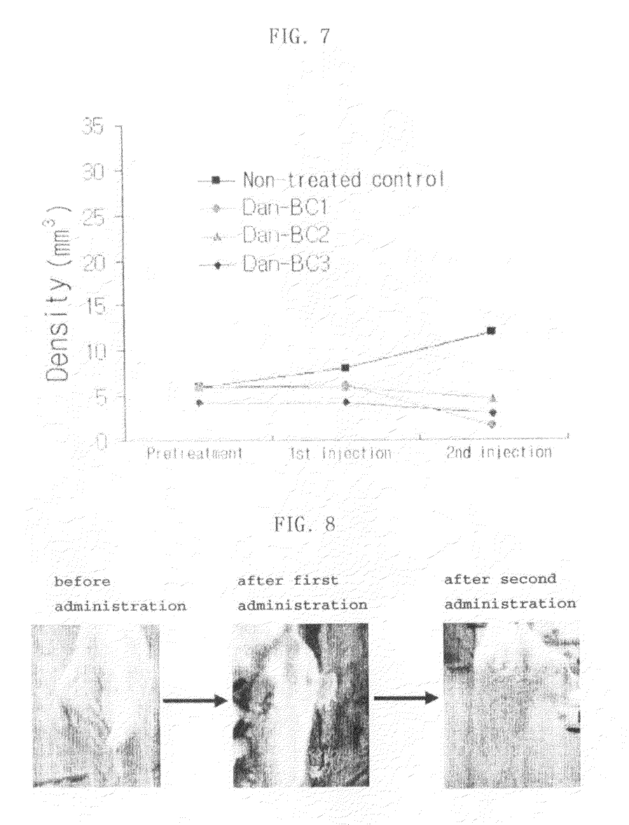

After administrating each of Dan-BC1, Dan-BC2 and Dan-BC3 compounds of the present invention weekly by 100 μM, respectively, into breast cancers naturally formed in female mouse transformed to form cancer, tumor inhibition effect was observed, each result of which is shown in FIGS. 8, 9 and 10, and FIG. 11.

As shown in FIG. 8, Dan-BC1 compound was administrated twice by 100 μM into transformed mouse in which breast cancer was naturally formed. In the case of Dan-BC1 compound, extinction of breast cancer was resulted even with administration of only once, thus, Dan-BC1 compound showed strong anticancer effect against breast cancer formed in the transformed mouse in vivo.

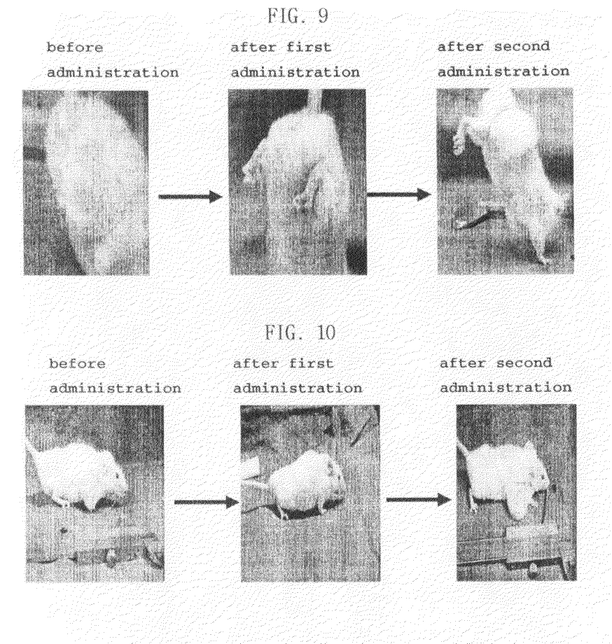

As shown in FIG. 9, after Dan-BC2 compound was administrated once by 100 μM into the transformed mouse to have the naturally formed breast cancer, breast cancer became extinct. In the case of Dan-BC2, the extinction of breast cancer was resulted even with only one administration, but formation of cancer was induced again at the time of the 2^{nd} injection. Dan-BC2 compound showed anticancer effect against breast cancer formed in transformed mouse in vivo.

As shown in FIG. 10, after Dan-BC3 compound was administrated once by 100 μM into the transformed mouse that breast cancer was naturally formed, breast cancer became extinct. In the case of Dan-BC3, extinction of breast cancer was resulted even with only one administration, but formation of cancer was derived again at the time of the second injection. Dan-BC3 compound showed anticancer effect against breast cancer formed in the transformed mouse to have breast cancer.

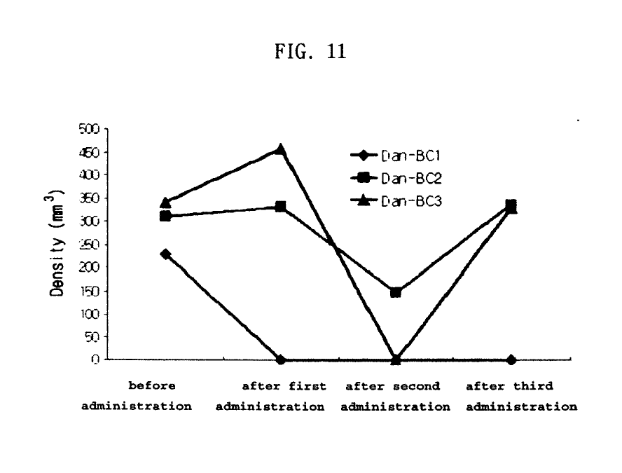

The aforementioned results were represented it in FIG. 11. As shown in FIG. 11, the effect of Dan-BC1 that is Triazine derivative according to the present invention on the transformed animal model having cancer was the most superior among Dan-BC1, Dan-BC2 and Dan-BC3, and as an order of the next Dan-BC3, and Dan-BC2, anti-cancer effect was detected.

The present invention provides Triazine derivative compound of formula (I) or pharmaceutically acceptable salt thereof, composition containing them, and therapeutic method using them useful for treatment of cancer.

1. A triazine derivative compound of formula (I): 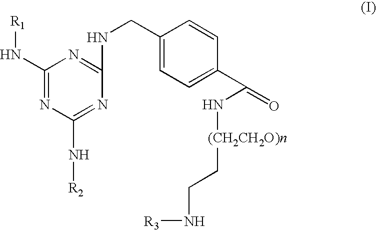
wherein R₁ is linear or branched alkyl of carbon number 1 to 8, R₂ is one selected from a group composed of linear branched alkyl, allyl, pyridinylmethyl, pyridinylethyl and pyridinylpropyl of carbon number 1 to 8, R₃ is a non-substituted benzoyl or substituted benzoyl by alkyl of carbon number 1 to 3, and n is integer of 1 to 3; or pharmaceutically acceptable salt thereof.

2. The triazine derivative compound and pharmaceutically acceptable salt thereof according to claim 1, wherein R₁ is butyl, R₂ is one selected from a group consisting of allyl, 1-ethylpropyl and pyridinylmethyl, R₃ is a non-substituted benzoyl, and n is 2.

3. The triazine derivative compound and pharmaceutically acceptable salt thereof according to claim 2, wherein R₂ is allyl.

4. A composition for treating cancer comprising the triazine derivative compound or pharmaceutically acceptable salt thereof of any one of claim 1.

5. A composition for treating cancer according to claim 4, wherein the cancer is selected from a group consisting of breast cancer, colorectum cancer, kidney cancer, brain cancer, ovarian cancer, stomach cancer, cervical cancer, lung cancer, leukemia, lymphoma, live cancer and uterine cancer.

6. A composition for treating cancer according to claim 5, wherein the cancer is breast cancer.

7. A composition for treating cancer comprising the triazine derivative compound or pharmaceutically acceptable salt thereof of any one of claim 2.

8. A composition for treating cancer comprising the triazine derivative compound or pharmaceutically acceptable salt thereof of any one of claim 3.
